(19) United States
(12) Patent Application Publication
(10) Pub. No.: US 2012/0035562 A1
(43) Pub. Date: Feb. 9, 2012

(54) SYSTEM AND METHOD FOR TREATING TISSUE OF A PATIENT USING A THERMOELECTRIC GENERATOR

(76) Inventors: Christopher Brian Locke, Bournemouth (GB); Richard Daniel John Coulthard, Verwood (GB)

(21) Appl. No.: 13/198,941

(22) Filed: Aug. 5, 2011

Related U.S. Application Data
(60) Provisional application No. 61/371,496, filed on Aug. 6, 2010.

(51) Int. Cl. A61M 1/00; H01R 43/00

(52) U.S. Cl. 604/319; 29/825

(57) ABSTRACT

A system and method for treating a tissue site of a patient may include applying a reduced pressure to a tissue site of a patient. Electricity may be generated in response to sensing a temperature level being generated by the patient. The generated electricity may be collected for use in applying a reduced pressure to the tissue site of the patient. In one embodiment, the generated electricity may be collected in a rechargeable battery.

### CROSS-REFERENCE TO RELATED APPLICATIONS

This application claims the benefit of and priority to U.S. Provisional Application No. 61/371,496 filed Aug. 6, 2010, which is hereby incorporated by reference.

### BACKGROUND

Technology of tissue treatment systems that provide reduced pressure for treating tissue sites, such as wounds, of patients has been significantly improved in recent years. Pump, dressing, and drape designs have all improved in quality, size reduction, and/or efficiency. While the above aspects of tissue treatment systems and components have improved, power source technology for powering pumps of the tissue treatment systems have only marginally improved. Even as newer pump designs have improved in reducing power draw, ultimately, a limiting factor of portable tissue treatment systems and non-portable tissue treatment systems is power supply duration and power consumption in general.

### SUMMARY

In addressing power supply issues for tissue treatment systems, the principles of the present invention provide for the use of an electricity generator, such as a thermoelectric generator (TEG), that generates electricity in response to sensing a temperature differential, where one temperature level is generated by a patient being treated by a tissue treatment and another temperature level is provided by ambient temperature or another coolant source, such as an ice pack. The electricity generated by the electricity generator may be used in powering the tissue treatment system by charging a power source, such as a rechargeable battery, thereby capturing previously lost energy that is freely available. The electricity generator may be configured in a variety of ways to accommodate dressing and tissue treatment system designs.

One embodiment of a system for treating a tissue site of a patient may include a dressing, a drape, a pump, and a thermoelectric generator. The drape may be configured to overlay said dressing and tissue site and form a seal with tissue surrounding the tissue site. The pump may be configured to generate reduced pressure at the tissue site. The thermoelectric generator may be configured to generate electricity in response to said thermoelectric generator sensing a temperature differential of a first temperature level and a second temperature level, where the first temperature level may be generated by the patient and the second temperature level may be generated by a coolant source. The generated electricity from the thermoelectric generator may be used to power the pump. The coolant source may be ambient temperature of a room in which the patient is located. The pump may be a disc pump, which is a form of a micro-pump.

One method for treating a tissue site of a patient may include applying a reduced pressure to a tissue site of a patient. Electricity may be generated in response to sensing a temperature level being generated by the patient. The generated electricity may be collected for use in applying a reduced pressure to the tissue site of the patient. In one embodiment, the generated electricity may be collected in a rechargeable battery.

One method of manufacturing a tissue treatment device may include providing a power source, electrically connecting an electrically powered device to the power source, and electrically connecting an electricity generator that is configured to generate electricity in response to sensing a temperature level generated by a patient to the power source and another temperature level from another temperature source. In one embodiment, electricity connecting an electricity generator may include connecting multiple electricity generators in series with the power source.

### BRIEF DESCRIPTION

Illustrative embodiments of the present invention are described in detail below with reference to the attached drawing figures, which are incorporated by reference herein and wherein:

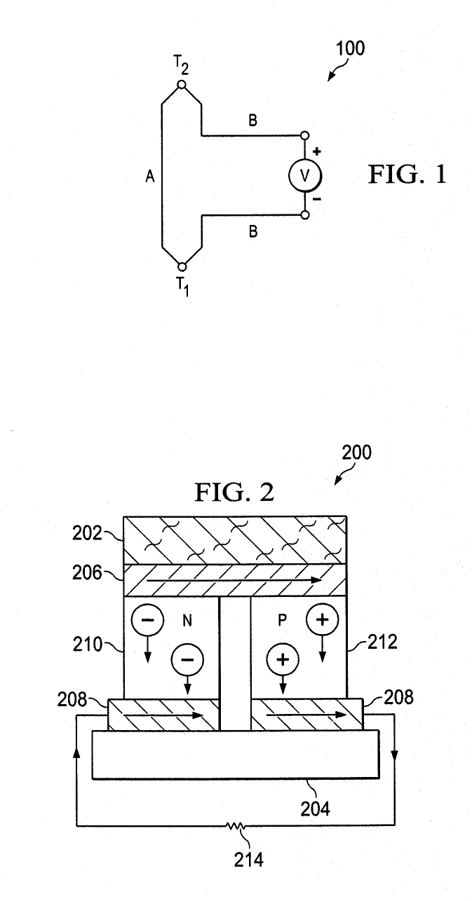

FIG. 1 is an illustration of an illustrative electronic circuit model of a traditional mechanical thermoelectric generator;

FIG. 2 is a schematic of an illustrative semiconductor thermoelectric generator;

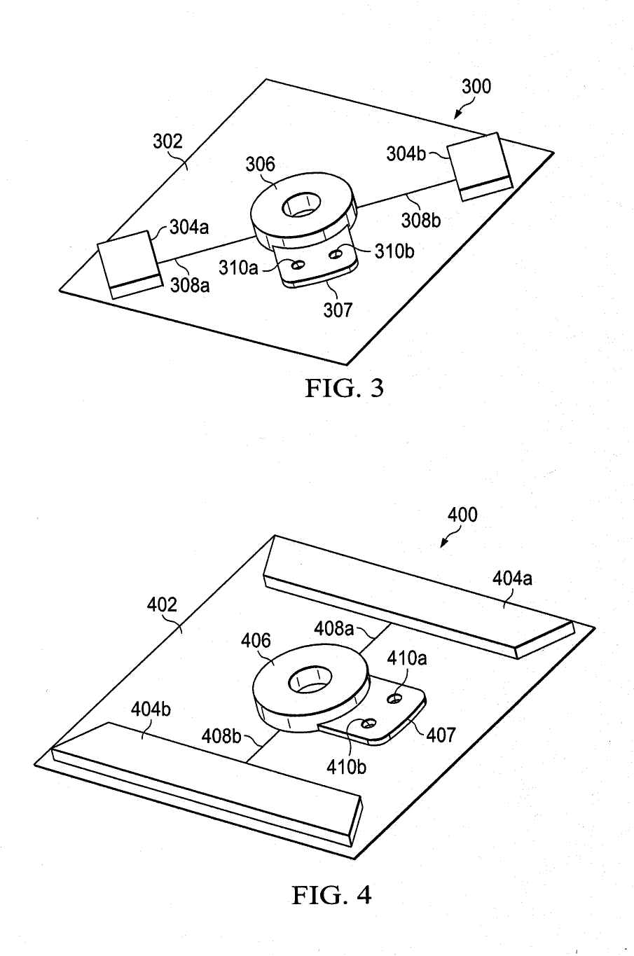

FIG. 3 is an illustration of an illustrative tissue treatment system including a drape for use in covering a tissue site of a patient and thermoelectric generators for use in charging a power source of a tissue treatment system;

FIG. 4 is an illustration of another illustrative tissue treatment system and drape for treating a tissue site of a patient;

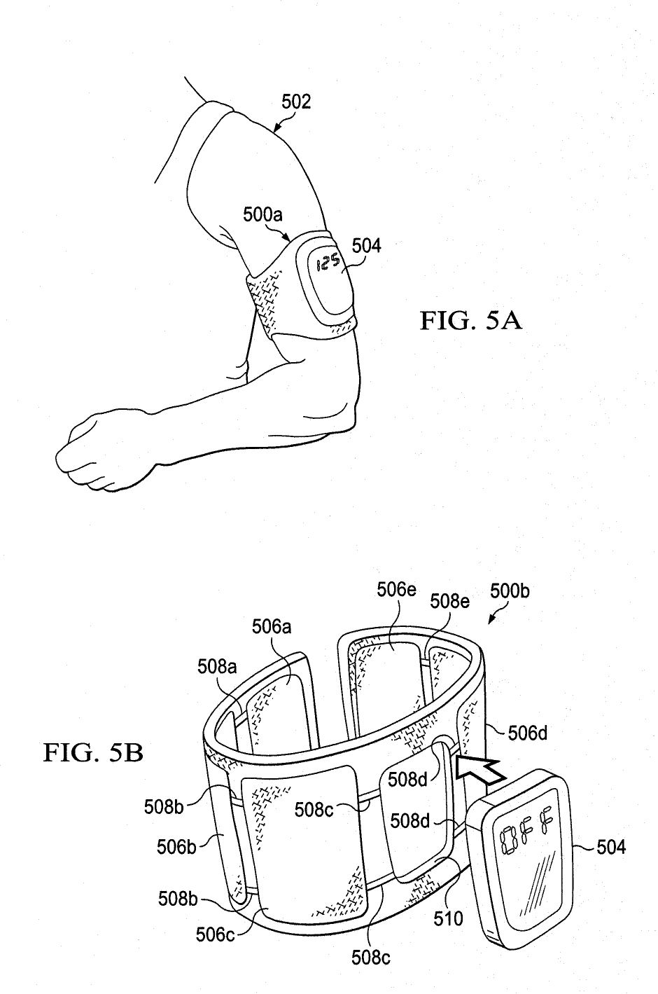

FIG. 5A is an illustration of a patient wearing an illustrative tissue treatment system combined with a band for use in treating a tissue site on an arm of the patient;

FIG. 5B is an illustration of an illustrative band configured with a tissue treatment system and thermoelectric generators integrated with the band;

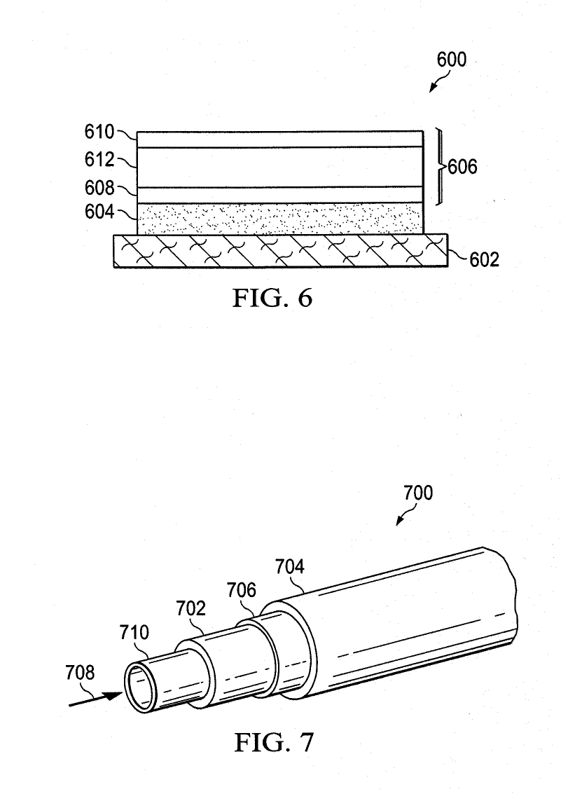

FIG. 6 is an illustration of an illustrative thermoelectric generator for use in generating electricity to power a power source of a tissue treatment system configured to treat a tissue site of a patient;

FIG. 7 is an illustration of a thermoelectric generator configured to operate as a tubular conduit for passing exudate fluid of a patient therethrough and generating electricity in response to the temperature of the exudate fluid;

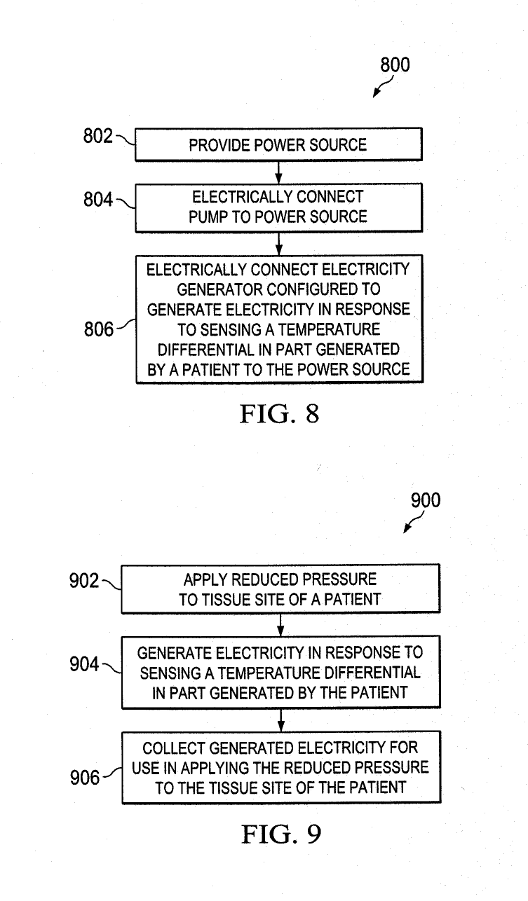

FIG. 8 is a flow diagram of an illustrative process for manufacturing a tissue treatment system with a thermoelectric generator; and

FIG. 9 is a flowchart of an illustrative process for treating a tissue site of a patient.

### DETAILED DESCRIPTION

FIG. 1 is a schematic of an illustrative circuit diagram model 100 of a traditional mechanical thermoelectric generator. Thermoelectric generators generate electricity based on the principles of the Seebeck effect, which is a process that converts temperature differentials directly into electricity. A temperature differential sensed by a thermoelectric generator produces a voltage or current. The temperature difference may be sensed by two different metals, or semiconductors in the case of a semiconductor design (see FIG. 2), which causes a continuous current to flow in the conductors if the conductors form a complete loop. The Seebeck effect has been used for many years to form thermocouples. Several thermocouples, when connected in series, are called a thermopile, which is constructed in order to increase the output voltage since the voltage induced over each individual thermocouple is small. The electrical model 100 is shown to include two temperature levels, T₁ and T₂. Metals A and B may be used to form an electrical loop to create a voltage, as shown. The voltage may be computed by using the Seebeck coefficients and temperature differential, as provided by the following equation: V=(SB−SA)(T₂−T₁), where SA and SB are the Seebeck coefficients or thermoelectric power of the metals A and B as a function of temperature, and T₁ and T₂ are the temperatures of the two junctions between the metals A and B.

With regard to FIG. 2, a schematic of an illustrative thermoelectric generator 200 represents a semiconductor thermoelectric generator that provides a more efficient and effective way to generate electricity from sensing temperature differentials than using different metals, as described in FIG. 1. As shown, a heat source 202, such as a patient's skin or tissue, may be used as a first temperature level and a cool side 204 that senses a second temperature level, such as a temperature level provided by ambient temperature, ice pack, or other temperature or coolant source, may provide a temperature differential with respect to the heat source 202 and be used to generate electricity by the thermoelectric generator 200. Between the heat source 202 and cool side 204, thermoconductive materials 206 and 208 may sandwich semiconductor material 210 and 212, which may include an n-type semiconductor material and p-type semiconductor material, respectively. In operation, the heat source 202 drives electrons in the n-type element toward the cool side 204, thus creating a current through the circuit. Holes in the p-type element flow in the direction of the current, thereby enabling the current to be used to power a load 214, and, thus, converting the thermal energy into electrical energy. In one embodiment, the load 214 may be a power source or part of a power source (e.g., electronic circuit) that is used to charge up the power source. In one embodiment, the power source may be a rechargeable battery and, optionally, include an electronic circuit used to receive electricity and recharge the rechargeable battery, as understood in the art. It should be understood that the configuration of the thermoelectric generator 200 is illustrative, and alternative configurations, such as changing the positions of the heat source 202 and cool side 204.

With regard to FIG. 3, an illustration of an illustrative tissue treatment system 300 is shown to include a drape 302 that is configured to overlay a tissue site of a patient that is being treated by the tissue treatment system 300. In one embodiment, the tissue treatment system may include one or more thermoelectric generators 304a-304b (collectively 304) that are used to generate electricity by sensing a temperature differential between a temperature level of tissue of a patient, for example, and another temperature level, such as ambient temperature of a room. The thermoelectric generators 304 are shown to include surfaces that are placed in contact with skin of a patient so as to operate at heat sources, while surfaces opposite the surfaces placed in contact with the skin may be used as cold sides so as to create a temperature differential to cause the thermoelectric generators 304 to generate electricity.

The tissue treatment system 300 may also include a pump unit 306, which, as shown, is a micro-pump or disc pump, as understood in the art, which operates to provide a reduced pressure at the tissue site. The reduced pressure at the tissue site, as understood in the art, helps generate tissue growth to improve or stimulate tissue healing. A control unit 307 may be configured with a processing unit or other control electronics (not shown) that controls and drives the pump unit 306 to generate the reduced pressure at the tissue site. The thermoelectric generators 304 may be electrically coupled to conductors 308a-308b (collectively 308) that conduct electricity generated by the thermoelectric generators 304 to the control unit 307. The control unit 307 may include one or more power sources 310a-310b (collectively 310) that may be used to drive power to the control unit 307 and pump unit 306. In one embodiment, the control unit 307, as part or separate from the power sources 310, may include circuitry (not shown) that, at least in part, utilizes electricity collected by the thermoelectric generators 304 by combining the collected electricity with the power sources 310 (e.g., by using an electronic summer) or to charge the power sources 310 while in operation and/or while not being used. In one embodiment, the power sources 310 are rechargeable batteries. In anther embodiment, the power sources 310 are capacitive elements. It should be understood that the principles of the present invention may be applied to any tissue treatment system that utilizes an electrically powered device, such as the pump unit 306.

As shown, the thermoelectric generators 304, pump unit 306, and control unit 307 may be configured to be small enough to be positioned within the confines of the drape 302. Alternatively, the configuration of the thermoelectric generators 304, pump unit 306, and control unit 307 may be positioned above the drape. In such an out-of-drape configuration, thermal conductors, such as surfaces of metal conductors, may extend through the drape so as to contact skin of the patient or, alternatively, reside on top of the drape and collect whatever heat passes through the drape for the heat source side of the thermoelectric generator. Other configurations in which the thermoelectric generators may be positioned at (i.e., under, above, integrated with, or near) the drape 302 may be utilized in accordance with the principles of the present invention. It should be understood that thermal conductors in a wide variety of configurations, rigid or flexible, may be utilized in accordance with the principles of the present invention. In one embodiment, the drape may be configured to allow the pump unit 306 to have at least a portion extend through the drape 302 with an airtight seal with the pump unit 306 so that fluid, such as air, may be discharged from beneath the drape 302 when sealed at a tissue site of a patient. In another embodiment in which the pump unit 306 is positioned on top of the drape 302, an inlet valve opening may be aligned with an opening (not shown) through the drape 302 to draw fluid from a pocket formed by the drape 302 and the patient. To reduce or eliminate the possibility of exudate fluid from entering the pump unit 306, a hydrophobic filter may be positioned in front of an inlet valve to prevent exudate from entering the pump unit 306. Additional description may be found in co-pending U.S. patent application Ser. No. 12/824,604 filed June 28, 2010, which is incorporated herein by reference in its entirety.

A variety of drape configurations are contemplated. As shown, the drape may be configured with conductors 308 that extend along a surface of the drape 302. The conductors 308 may be positioned on top, bottom, or through the drape 302. In one embodiment, the conductors are printed on the drape 302. The thermoelectric generators 304 may be fixedly attached or removably attached to the drape 302. If fixedly attached, the thermoelectric generators 304 may be disposable so that upon completed use of the drape 302, the drape 302 and thermoelectric generators 304 are disposed with other biomedical waste. If removably attached, the thermoelectric generators 304 are formed in a manner so as to be washable and capable of being sterilized for re-use with the same or other patients. In the thermoelectric generators 304 are removable, then the drape 302 may be configured with connectors (not shown) onto which the thermoelectric generators 304 may be attached to provide power via the conductors 308 to the pump unit 306. The connectors may be snaps, clips, or other conductive connector onto which the thermoelectric generators 304 may be attached for securing to the drape 302 and deliver electricity that has been generated. The pump unit 306 may also be fixedly or temporarily attached to the drape 302 in the same or similar manner as the thermoelectric generators 304.

With regard to FIG. 4, an illustrative tissue treatment system 400 may include a drape 402 that has thermoelectric generator film sheets 404a and 404b (collectively 404) that may be attached or applied to the drape 402. Although shown as two film sheets 404, other numbers of film sheets may be utilized. The film sheets 404 may be single or multiple layers of sheets, and may be metallic or other material capable of thermal conduction. The film sheets 404 may have additional materials and/or electrical components that are capable of generating electricity, as described with regard to FIGS. 1 and 2. The film sheets 404 may be adhered or otherwise attached to the skin-side surface of the drape 402. The tissue treatment system 400 may also include a pump unit 406 and control unit 407 that are used to generate reduced pressure at a tissue site for promoting tissue growth of the tissue site, as understood in the art. Conductor lines 408a and 408b (collectively 408) may provide for electricity to be carried between the thermoelectric generator film sheets 404a and 404b to the control unit 407 for use in providing electricity or power to power sources 410a and 410b, as described with regard to FIG. 3. It should be understood that the power sources 410a and 410b may be a single power source or multiple power sources that are recharged using the same or different circuitry and that the conductor lines 408 may be configured to cause the power sources to operate in series.

With regard to FIG. 5A, an illustration of a band 500a is shown to be wrapped around an arm of a patient 502. The band 500a is further shown to be configured with a tissue treatment system 504 that is used to generate reduced pressure for treating a tissue site of the patient 502. The band 500a may be configured with thermoelectric generators (not shown) that sense temperature of the patient's arm at or near the tissue site to generate electricity in response to a differential temperature of the tissue of the patient's arm and a second temperature level, which may be ambient temperature or any other temperature level (e.g., ice pack in contact with the thermoelectric generators). Being near or local to a tissue site may include being on a body part on which a tissue site exists (e.g., arm, leg, abdomen, etc.). It should be understood that the band 500a may be configured to be wrapped around any body part. It should further be understood that the band 500a may alternatively be configured more in the fashion of a bandage with adhesive for sticking to a patient as opposed to wrapping around a patient, thereby enabling an easier installation for a patient's torso.

With regard to FIG. 5B, an illustration of an illustrative band 500b is shown to include a tissue treatment system 504 and thermoelectric generators 506a-506d (collectively 506) and conductors 508a-508e (collectively 508) attached to the band 500b. The conductors 508 are utilized for conducting electricity generated by the thermoelectric generators 506 in series or in parallel to the tissue treatment system 504. The tissue treatment system 504 may include a power source (not shown) that is used to power the tissue treatment system 504 and, in response to receiving electricity from the thermoelectric generators 506 via the conductors 508, charge the power source. An opening 510 in the band 500b may be configured to receive the tissue treatment system 504. In the same or analogous manner, the band 500b may include openings (not shown) into which the thermoelectric generators are positioned that allow one side of the thermoelectric generators to directly contact tissue (e.g., skin) or indirectly contact the tissue (e.g., via foam, gauze, mesh, or any other material) of a patient and another side to be exposed to ambient temperature or other differential temperature source (e.g., ice pack). Alternatively, the thermoelectric generators may be positioned within pockets in the band 500b that allow for differential temperature sensing to cause electricity to be generated. The tissue treatment system 504 may alternatively be fixedly attached to the band 500b, and the thermoelectric generators 506 may be fixedly attached to the band 500b, as well. Alternatively, the thermoelectric generators 506 and tissue treatment system 504 may be temporarily attached to the band 500b via electrically conductive connectors, such as snaps (not shown), which may be in electrical communication with the conductors 508 for providing electricity flow between the thermoelectric generators 506 and tissue treatment system 504 for use in powering a pump by charging a battery or otherwise, as described herein. Other mechanical fastening mechanisms may be utilized to secure the thermoelectric generators 506 to the band 500b.

With regard to FIG. 6, an illustration of an illustrative tissue site 600 is shown to include tissue 602 of a patient having a wound dressing member 604, such as a foam dressing, disposed thereon with a thermoelectric generator 606 being disposed on top of the wound dressing member 604. In this embodiment, the wound dressing member 604 operates to collect exudate from the tissue 602, thereby conducting heat to the thermoelectric generator 606. If a drape is included, then the drape may be disposed above or below the thermoelectric generator 606. If the drape is positioned below the thermoelectric generator 606, then a vent that provides for an air tight seal with the thermoelectric generator 606 may provide for reduced pressure at the tissue 602 to be accomplished by use of a vacuum pump, for example, and direct thermal contact to be made between the wound dressing member 604 and the thermoelectric generator 606. Although the thermoelectric generator 606 is not directly in contact with the tissue 600 of the patient, the foam dressing 604 and exudate fluid that is absorbed into the wound dressing member 604 is capable of operating as a heat transfer element to transfer heat produced by the patient to a “hot” side 608 of the thermoelectric generator 606. The thermoelectric generator 606 may also include a “cold” side 610 that senses ambient temperature of a room or other temperature source. A semiconductor material 612 may provide for electricity generation by sensing the temperature differential between the hot side 608 and cold side 610 of the thermoelectric generator 606, as described with regard to FIG. 2. By having the thermoelectric generator 606 disposed on top of the foam 604, the tissue treatment system may be more compact than if disposed near the tissue site, since being near the tissue site would cause the thermoelectric generator 606, which is part of the tissue treatment system, to have more horizontal area than if disposed on top of the foam 604. Being near the tissue site means to be disposed at surrounding tissue from a tissue site (e.g., wound) that is being treated with a drape covering.

In an alternative embodiment, the exudate fluid that is collected may be applied to one or more chemicals, such as iron or copper salts, that produce an exothermic reaction to provide a heat source for the thermoelectric generator 606. The application of the exudate fluid may simply include adding the exudate fluid to a canister with the chemical(s). In one embodiment, the exudate fluid may be mechanically mixed with the chemical(s). In one embodiment, the exothermic reaction may be performed in a canister (not shown) or in-line, as shown in FIG. 6. Being in-line means that the thermoelectric generator is positioned in a flow path of exudate fluid, which may include being positioned above a tissue site, as shown in FIG. 6. In one embodiment, the thermoelectric generator may be formed in a particular shape that forms the in-line flow path or conduit, such as being configured in a tubular form or any other shape, through which the exudate fluid flows so that the exudate fluid has an opportunity to interact with the thermoelectric generator. In the case of the thermoelectric generator being in a tubular form or configured to fit within a tube, the thermoelectric generator may be positioned as an inside member so that an outside member may operate to contact a coolant source, thereby allowing for a temperature differential. In the case of the thermoelectric generator using a reagent, such as a chemical, various structures, such as being disposed within a foam, mesh, or other porous material, may be utilized to allow the exudate to contact the reagent along the exudate flow path. Being in a canister may include being fixedly or non-fixedly positioned in a collection canister for the exudate fluid. It should be understood that a variety of different canister and in-line configurations for integrating the exudate fluid with the chemicals may be utilized to produce heat that is used to generate electricity utilizing a thermoelectric generator in accordance with the principles of the present invention.

With regard to FIG. 7, an illustration of a thermoelectric generator 700 configured in the shape of a conduit, in this case a tubular conduit, is shown. The thermoelectric generator 700 may have a hot core 702 surrounded by a cold face 704. Between the hot core 702 and cold face 704, semiconductor material 706 that is appropriately doped, as described with regard to FIG. 2, may be utilized to generate electricity as a result of exudate fluid 708 or wound fluid that is hotter than the ambient temperature or other temperature being sensed by the cold face 704. In one embodiment, a disposable fuel core 710 may be used to protect the hot core 702 from becoming contaminated by patients. The disposable fuel core 710 may have a thermal coefficient that allows the temperature level of the exudate fluid 708 to be sensed by the hot core 702, thereby providing a temperature differential and causing the thermoelectric generator to generate electricity for use by the tissue treatment system in generating reduced pressure to a tissue site. Although not shown, the thermoelectric generator 700 may be electrically attached to a tissue treatment system that uses a pump that delivers reduced pressure at a tissue site via conduit(s) and removes fluid from the tissue site. Electrical energy produced by the thermoelectric generator 700 may reduce power consumption of the tissue treatment system.

With regard to FIG. 8, a flow chart of an illustrative process 800 for manufacturing a tissue treatment system is shown. The process 800 starts at step 802, where a power source is provided. The power source may include a rechargeable battery, energy storage unit, such as a capacitor, or any other power source, including an AC-to-DC converter to convert power being delivered from a wall socket, as understood in the art. At step 804, a pump may be electrically connected to the power source. It should be understood that rather than using a pump, any electrically powered device used to treat tissue of a patient may be utilized. At step 806, an electricity generator configured to generate electricity in response to sensing a temperature differential in part generated by a patient may be electrically connected to the power source. By electrically connecting the electricity generator to the power source, electricity generated by the electricity generator may be used to provide additional power to the power source during active operation or charge the power source either while the tissue treatment system is operating or charge the power source while not in operation. Although described herein as generating electricity for use in applying a reduced pressure, it should be understood that the generated electricity be used to power components other than a pump, such as a clock, computing unit, electronic display, and/or other electronic components(s), and still be considered to be used in applying a reduced pressure since additional power will be available for the pump that would otherwise be used for powering the other component(s).

With regard to FIG. 9, a flow chart of an illustrative process 900 of a tissue treatment system for treating a tissue site of a patient is shown. At step 902, reduced pressure may be applied to the tissue site of a patient. In applying the reduced pressure to the tissue site, a pump may be utilized to reduce pressure at the tissue site that is covered by a drape, as understood in the art. At step 904, electricity may be generated in response to sensing a temperature differential in part generated by the patient. In one embodiment, the patient's skin or other tissue that provides heat may be sensed by a thermoelectric generator to form a temperature differential between the temperature level of the patient's tissue and other temperature or coolant source, such as ambient room temperature, ice pack, or other coolant source. At step 906, the generated electricity may be collected for use in applying the reduced pressure to the tissue site of the patient. The collected electricity may be collected in a power source, such as a rechargeable battery or capacitor, and be used to power a pump, such as a micro-pump, that is being used to reduce pressure at the tissue site of the patient.

The previous detailed description is of a small number of embodiments for implementing the invention and is not intended to be limiting in scope. One of skill in this art will immediately envisage the methods and variations used to implement this invention in other areas than those described in detail. For example, rather than applying thermoelectric generators to a reduced pressure tissue treatment system, alternative treatment systems, for tissue or otherwise, may utilize thermoelectric generators to generate electricity for use in powering the system. The following claims set forth a number of the embodiments of the invention disclosed with greater particularity. Although a number of independent embodiments have been described, any features of any of the embodiments may be combined or exchanged with features of any other embodiments.

1. A system for treating a tissue site of a patient, said system comprising:
a pump configured to generate reduced pressure at the tissue site; and
a thermoelectric generator configured to generate electricity in response to said thermoelectric generator sensing a temperature differential of a first temperature level and a second temperature level, the first temperature level being generated by the patient and the second temperature level being generated by a coolant source, the generated electricity being used to power said pump.

2. The system according to claim 1, further comprising a power source local to said pump and in electrical communication with said thermoelectric generator, said power source being configured to receive the generated electricity for use in powering said pump.

3. The system according to claim 2, wherein said power source is a rechargeable battery.

4. The system according to claim 1, wherein said pump is a micro-pump.

5. The system according to claim 1, wherein said thermoelectric generator is configured to be in contact with tissue of the patient on a first side of the thermoelectric generator.

6. The system according to claim 1, wherein said thermoelectric generator is configured to be a tubular shape, and wherein an inner surface of the tubular shape is configured as a “hot” surface and capable of allowing exudate from the patient to pass therethrough, thereby causing said thermoelectric generator to generate electricity.

7. The system according to claim 1, wherein said thermoelectric generator is connected to a drape.

8. The system according to claim 7, wherein said thermoelectric generator includes multiple sections that are distinctly positioned on the drape.

9. The system according to claim 7, wherein said thermoelectric generator is temporarily connected to the drape.

10. The system according to claim 1, further comprising a band configured to wrap around a body part, said band being configured to attach said thermoelectric generator thereto.

11. The system according to claim 10, wherein said band is further configured to attach said pump thereto.

12. The system according to claim 1, wherein said thermoelectric generator is a semiconductor thermoelectric generator.

13. A method for treating a tissue site of a patient, said method comprising:
applying a reduced pressure to a tissue site of a patient;
generating electricity in response to sensing a temperature level being generated by the patient; and
collecting the generated electricity for use in applying a reduced pressure to the tissue site of the patient.

14. The method according to claim 13, wherein collecting the generated electricity includes collecting the generated electricity in a power source local to the tissue site of the patient.

15. The method according to claim 14, wherein collecting the generated electricity in a power source includes collecting the generated electricity in a rechargeable battery.

16. The method according to claim 13, wherein generating electricity includes contacting a thermoelectric generator to tissue of the patient.

17. The method according to claim 16, further comprising positioning the thermoelectric generator between a drape covering the tissue site and tissue of the patient.

18. The method according to claim 13, wherein generating electricity includes passing exudate fluid of the patient across a thermoelectric generator.

19. A method of manufacturing a tissue treatment device, said method comprising:
providing a power source for the tissue treatment device;
providing an electrically powered device to the power source, the electrically powered device being configured to pump fluid;
electrically connecting an electrically powered device to the power source; and
electrically connecting an electricity generator that is configured to generate electricity in response to sensing a temperature level generated by a patient to the power source and another temperature level.

20. The method according to claim 19, wherein providing a power source includes providing a rechargeable battery.

21. The method according to claim 19, further comprising mechanically connecting the electricity generator to a drape used to seal a tissue site of the patient to enable the electrically powered device to apply reduced pressure to the tissue site.

22. The method according to claim 19, further comprising mechanically connecting the electrically powered device, power source, electricity generator to a band configured to wrap around a body part of the patient and cause the electricity generator to touch the body part of the patient.

23. The method according to claim 19, wherein electronically connecting an electricity generator includes electrically connecting a semiconductor thermoelectric generator.

24. The method according to claim 19, further comprising increasing the temperature level generated by a patient by applying exudate fluid from the patient to a chemical to cause an exothermic reaction.

25. A system for treating a tissue site of a patient using reduced pressure, said system comprising:
a pump configured to generate reduced pressure at the tissue site; and
a thermoelectric generator configured to generate electricity in response to the thermoelectric generator sensing a temperature differential of a first temperature level and a second temperature level, the first temperature level being generated by the patient, the generated electricity being supplied to a system utilized to supply the reduced pressure.

26. The system according to claim 25, further comprising an electrically powered device for supplying reduced pressure to the tissue site, the electrically powered device including the pump, wherein the electricity from the thermoelectric generator is utilized to at least partially power the pump.

27. The system according to claim 25, further comprising an electrically powered device for supplying reduced pressure to the tissue site, the electrically powered device including the pump and a rechargeable power source, wherein the electricity from the thermoelectric generator is utilized to recharge the power source.

28. The system according to claims 25, wherein a first side of the thermoelectric generator is configured to be in contact with tissue of the patient.

29. The system according to claim 25, wherein the second temperature level is provided by a coolant source.

30. The system according to claim 25, wherein the second temperature is provided by exudate from the tissue site.

31. The system according to claim 30, wherein the exudate is reacted with a reagent such that an exothermic reaction occurs.

32. The system according to claim 25, further comprising a drape for creating a sealed space at the tissue site, wherein the thermoelectric generator is at least partially located on the drape.

33. The system according to claim 32, wherein the thermoelectric generator includes at least one thermoelectric generator sheet positioned on the drape.

34. The system according to claim 25, further comprising a dressing member, wherein the thermoelectric generator is located on the dressing member.

35. The system according to claim 25, wherein the thermoelectric generator is configured to be a tubular shape to provide a conduit for exudate from the tissue site, the inner surface of the conduit sensing the first temperature level, and an outer surface of the conduit sensing the second temperature level.

36. The system according to claim 35, wherein the thermoelectric generator further comprising a disposable core to protect the thermoelectric generator from the exudate.

37. The system according to claim 25, further comprising a band configured to wrap around a body part, the band being configured to attach the thermoelectric generator thereto.

38. The system according to claim 37, wherein the band is further configured to attach the pump thereto for providing reduced pressure to the tissue site.

39. The system according to claim 25, wherein the thermoelectric generator is a semiconductor thermoelectric generator.
